(19) United States
(12) Patent Application Publication
(10) Pub. No.: US 2024/0299340 A1
(43) Pub. Date: Sep. 12, 2024

(54) COMPOSITION COMPRISING AT LEAST ONE NATURAL OR SYNTHETIC DIBENZO-ALPHA-PYRONE AS ACTIVE SUBSTANCE AND ITS MANUFACTURING PROCESS

(71) Applicants: ELEONOR, Waterloo (BE); TILMAN, Baillonville (BE)

(72) Inventors: Cristina LOIRA-PASTORIZA, Bruxelles (BE); Fabian PRIEM, Waterloo (BE); Yvan DIERCKXSENS, Overijse (BE)

(73) Assignees: ELEONOR, Waterloo (BE); TILMAN, Baillonville (BE)

(21) Appl. No.: 18/594,732

(22) Filed: Mar. 4, 2024

(30) Foreign Application Priority Data
Mar. 6, 2023 (BE) 2023/5164

(51) Int. Cl. A61K 31/352; A61K 9/10

(52) U.S. Cl. CPC A61K 31/352; A61K 9/10

(57) ABSTRACT

Compositions in the form of a thermoformed extrudate, possibly packaged in pellet, flake, granule, powder, effervescent tablet or not, in injectable solution or not, in drink, in suspension, in gel, in ointment or in any other suitable form allowing administration to an animal or a human being, comprising at least one natural or synthetic dibenzo-α-pyrone as active substance and at least one natural or synthetic support.

The present invention relates to a composition comprising at least one natural or synthetic dibenzo-α-pyrone as active substance, to its manufacturing process and to its use.

In the sense of the present invention, the terms “dibenzo-α-pyrone” and “dibenzo-alpha-pyrone” are synonyms.

According to the invention, a natural or synthetic dibenzo-α-pyrone is a compound, of which the basic chemical structure is as follows (General formula (I)):

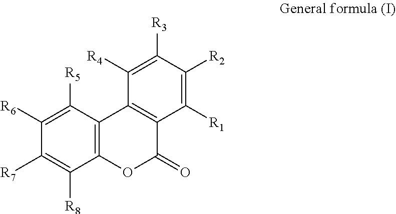

wherein,
R₁ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₂ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₃ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₄ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₅ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₆ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₇ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₈ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl.

In particular, according to an embodiment according to the present invention, R₁ preferably represents a hydrogen, a C₁₋₄ alkyl, OR′, OSO₃R′, C(═O)R′, OC(═O)R′, a halogen, an O-glucoside, or a C-glucoside; said C₁₋₄ alkyl being optionally substituted by one or more hydroxys; R′ representing a hydrogen or a C₁₋₄ alkyl.

Preferably, R₁ represents a hydrogen, OR′, a halogen, an O-glucoside, or a C-glucoside; R′ representing a hydrogen or a C₁₋₄ alkyl.

More preferably, R₁ represents a hydrogen, or OR′; R′ representing a hydrogen or a C₁₋₄ alkyl.

More preferably, R₁ represents a hydrogen, OH or OCH₃.

In particular, according to an embodiment according to the present invention, R₂ preferably represents a hydrogen, a C₁₋₄ alkyl, OR′, OSO₃R′, C(═O)R′, OC(═O)R′, a halogen, an O-glucoside, or a C-glucoside; said C₁₋₄ alkyl being optionally substituted by one or more hydroxys; R′ representing a hydrogen or a C₁₋₄ alkyl.

Preferably, R₂ represents a hydrogen, a C₁₋₄ alkyl, OR′, a halogen, an O-glucoside, or a C-glucoside; said C₁₋₄ alkyl being optionally substituted by one or more hydroxys; R′ representing a hydrogen or a C₁₋₄ alkyl.

More preferably, R₂ represents a hydrogen, a C₁₋₄ alkyl, OR′, or an O-glucoside; said C₁₋₄ alkyl being optionally substituted by one or more hydroxys; R′ representing a hydrogen or a C₁₋₄ alkyl.

More preferably, R₂ represents a hydrogen, CH₃, OH, OCH₃ or an O-glucoside.

In particular, according to an embodiment according to the present invention, R₃ preferably represents a hydrogen, a C₁₋₄ alkyl, OR′, OSO₃R′, C(═O)R′, OC(═O)R′, a halogen, an O-glucoside, or a C-glucoside; said C₁₋₄ alkyl being optionally substituted by one or more hydroxys; R′ representing a hydrogen or a C₁₋₄ alkyl.

Preferably, R₃ represents a hydrogen, a C₁₋₄ alkyl, CH(═O), C(═O)OR′, OR′, OSO₃R′, C(═O)R′, OC(═O)R′, a halogen, an O-glucoside, or a C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl.

More preferably, R₃ represents a hydrogen, OR′, OSO₃R′, C(═O)R′, a halogen, an O-glucoside, or a C-glucoside; said O-dihydrobenzofurane being optionally substituted by one or more groups chosen from among OR′ or CH₂OR′; R′ representing a hydrogen or a C₁₋₄ alkyl.

More preferably, R₃ represents a hydrogen, OH, OCH₃, C(═O)C_{1-4 alkyl}, OSO₃H or an O-dihydrobenzofurane substituted by one or more groups chosen from among OH or CH₂OH.

In particular, according to an embodiment according to the present invention, R₄ preferably represents a hydrogen, a C₁₋₄ alkyl, OR′, OSO₃H, C(═O)R′, OC(═O)R′, a halogen, an O-glucoside, or a C-glucoside; said C₁₋₄ alkyl being optionally substituted by one or more hydroxys; R′ representing a hydrogen or a C₁₋₄ alkyl.

Preferably, R₄ represents a hydrogen, OR′, a halogen, an O-glucoside, or a C-glucoside; R′ representing a hydrogen or a C₁₋₄ alkyl.

More preferably, R₄ represents a hydrogen, or OR′; R′ representing a hydrogen or a C₁₋₄ alkyl.

More preferably, R₄ represents a hydrogen, OH or OCH₃.

In particular, according to an embodiment according to the present invention, R₅ preferably represents a hydrogen, a C₁₋₄ alkyl, OR′, OSO₃H, C(═O)R′, OC(═O)R′, a halogen, an O-glucoside, or a C-glucoside; said C₁₋₄ alkyl being optionally substituted by one or more hydroxys; R′ representing a hydrogen or a C₁₋₄ alkyl.

Preferably, R₅ represents a hydrogen, a C₁₋₄ alkyl, OR′, a halogen, an O-glucoside, or a C-glucoside; said C₁₋₄ alkyl being optionally substituted by one or more hydroxys; R′ representing a hydrogen or a C₁₋₄ alkyl.

More preferably, R₅ represents a hydrogen, a C₁₋₄ alkyl, or OR′; said C₁₋₄ alkyl being optionally substituted by one or more hydroxys; R′ representing a hydrogen or a C₁₋₄ alkyl.

More preferably, R₅ represents a hydrogen, CH₃, CH₂OH, on OH.

In particular, according to an embodiment according to the present invention, R₆ preferably represents a hydrogen, a C₁₋₄ alkyl, OR′, OSO₃H, C(═O)R′, OC(═O)R′, a halogen, an O-glucoside, or a C-glucoside; said C₁₋₄ alkyl being optionally substituted by one or more hydroxys; R′ representing a hydrogen or a C₁₋₄ alkyl.

Preferably, R₆ represents a hydrogen, OR′, a halogen, an O-glucoside, or a C-glucoside; R′ representing a hydrogen or a C₁₋₄ alkyl.

More preferably, R₆ represents a hydrogen, or OR′, or a halogen; R′ representing a hydrogen or a C₁₋₄ alkyl.

More preferably, R₆ represents a hydrogen, OH or OCH₃ or Cl.

In particular, according to an embodiment according to the present invention, R₇ preferably represents a hydrogen, a C₁₋₄ alkyl, OR′, OSO₃H, C(═O)R′, OC(═O)R′, a halogen, an O-glucoside, or a C-glucoside; said C₁₋₄ alkyl being optionally substituted by one or more hydroxys; R′ representing a hydrogen or a C₁₋₄ alkyl.

Preferably, R₇ represents a hydrogen, OR′, OSO₃H, a halogen, an O-glucoside, or a C-glucoside; R′ representing a hydrogen or a C₁₋₄ alkyl.

Preferably, R₇ represents a hydrogen, OR′, OSO₃H, a halogen, an O-glucoside, or a C-glucoside; R′ representing a hydrogen or a C₁₋₄ alkyl.

More preferably, R₇ represents a hydrogen, OH, OCH₃, an O-glucoside, or OSO₃H.

In particular, according to an embodiment according to the present invention, R₈ preferably represents a hydrogen, a C₁₋₄ alkyl, OR′, OSO₃H, C(═O)R′, OC(═O)R′, a halogen, an O-glucoside, or a C-glucoside; said C₁₋₄ alkyl being optionally substituted by one or more hydroxys; R′ representing a hydrogen or a C₁₋₄ alkyl.

Preferably, R₈ represents a hydrogen, a C₁₋₄ alkyl, OR′, a halogen, an O-glucoside, or a C-glucoside; said C₁₋₄ alkyl being optionally substituted by one or more hydroxys; R′ representing a hydrogen or a C₁₋₄ alkyl.

More preferably, R₈ represents a hydrogen, a C₁₋₄ alkyl, or OR′, or a halogen; said C₁₋₄ alkyl being optionally substituted by one or more hydroxys; R′ representing a hydrogen or a C₁₋₄ alkyl.

More preferably, R₈ represents a hydrogen, CH₃, OH, OCH₃ or Cl.

According to preferred embodiments according to the present invention, dibenzo-α-pyrone is preferably a compound, the basic chemical structure of which is chosen from among one of the follow (General formulas (II) to (VIII))

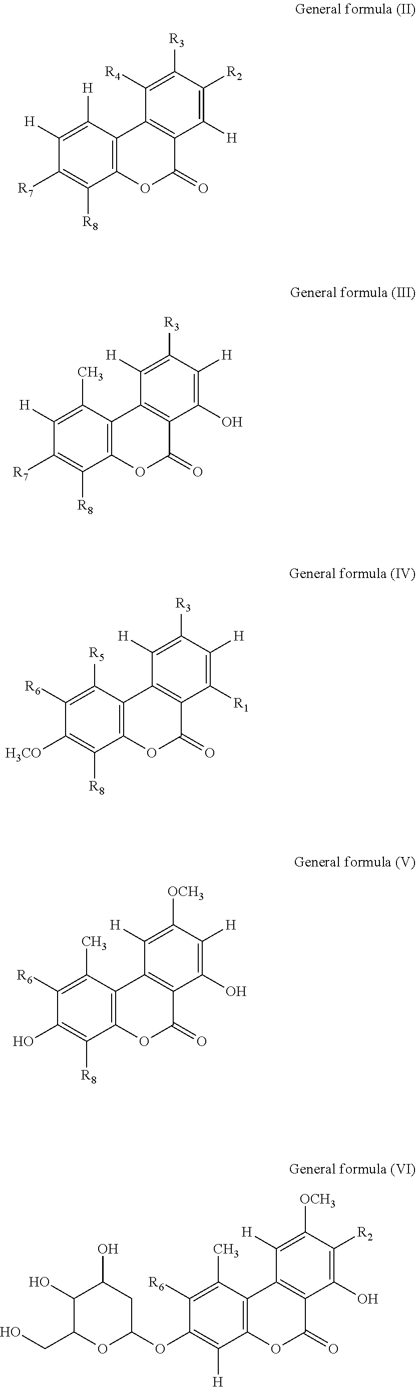

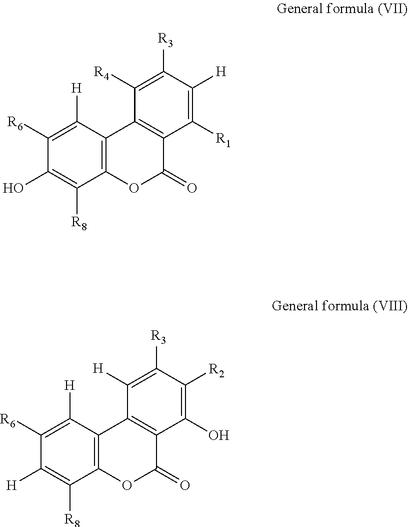

wherein, R₁, R₂, R₃, R₄, R₅, R₆, R₇, and R₈ are such as defined above for the general formula (I).

According to a preferred embodiment according to the present invention, said dibenzo-α-pyrone according to formula (II) is a compound chosen from among urolithin A, isourolithin A, urolithin B, urolithin C, urolithin D, urolithin E, urolithin M-5, urolithin M-6, urolithin M-7, isourolithin B, urolithin A glucuronide, urolithin B glucuronide, 8-Omethylurolithin A, 8,9-di-O-methylurolithin C, and 8,9-di-O-methylurolithin D.

Preferably, according to the present invention, said dibenzo-α-pyrone according to formula (II) is a compound chosen from among urolithin A, isourolithin A, urolithin B, isourolithin B, urolithin C, urolithin D, urolithin E, urolithin A glucuronide, and urolithin B glucuronide.

More preferably, according to the present invention, said dibenzo-α-pyrone according to formula (II) is a compound chosen from among urolithin A, isourolithin A, and urolithin B.

According to a preferred embodiment according to the present invention, said dibenzo-α-pyrone according to formula (III) is a compound chosen from among alternariol, alternariol 9-methyl ether, 3-O-alternariol sulfate 9-methyl ether, 9-0 alternariol sulfate, and 4-hydroxyalternariol 9-methyl ether.

According to a preferred embodiment according to the present invention, said dibenzo-α-pyrone according to formula (IV) is a compound chosen from among graphislactone A, graphislactone B, graphislactone C, graphislactone E, and graphislactone F, graphislactone G, and graphislactone H.

According to a preferred embodiment according to the present invention, said dibenzo-α-pyrone according to formula (V) is a compound chosen from among palmariol A and palmariol B.

According to a preferred embodiment according to the present invention, said dibenzo-α-pyrone according to formula (VI) is a compound chosen from among lysilactone A, lysilactone B, and lysilactone C.

According to a preferred embodiment according to the present invention, said dibenzo-α-pyrone according to formula (VII) is a compound chosen from among altertenuol, fasciculiferol.

According to a preferred embodiment according to the present invention, said dibenzo-α-pyrone according to formula (VIII) is a compound chosen from among sabilactone and murylactone.

The term “alkyl”, used individually or in combination, is defined as being a radical derived from an alkane comprising, unless otherwise indicated, from 1 to 4 carbon atoms. For example, a C_{F-G}alkyl defines an unbranched or branched alkyl radical containing from F to G carbon atoms, for example a C₄ alkyl defines an unbranched or branched alkyl containing from 1 to 4 carbons such as by example, methyl, ethyl, 1-propyl, 2-propyl, 1-butyl, 2-butyl, 2-methyl-1-propyl. An alkyl group can be branched or unbranched. The branched or unbranched alkyl chain can be linked at any position as long as a stable compound is obtained.

The term “halogen”, used individually or in combination, is defined as meaning all halogens, i.e. chloro (Cl), bromo (Br), fluoro (F) and iodo (I) groups.

The term “glucoside”, used individually or in combination, is defined as being a compound, wherein at least one saccharide is linked to another functional group by a glucoside bond. The glucoside chain can comprise from 1 to 4 saccharides.

The term “glucoside bond”, used individually or in combination, is defined as being a bond formed between a hemiacetal group of a saccharide and a functional group of a compound. Said functional group can be an OH (O-glucoside) group or a —CR₁R₂R₃ (C-glucoside) group.

The terms “acyl O-glucoside” or “acyl C-glucoside”, used individually or in combination, are defined as being a compound, wherein at least one hydroxyl of the glucoside chain is esterified by an organic acid, such as, for example, acetic acid, benzoic acid, cinnamic acids (such as caffeic acid, ferulic acid, or p-coumaric acid) and/or phenylpropanoic acid.

The terms “sulfate O-glucoside” or “sulfate C-glucoside”, used individually or in combination, are defined as being a compound, wherein at least one hydroxyl of the glucoside chain is esterified by sulfuric acid.

In the sense of the present invention, the term “natural or synthetic dibenzo-α-pyrone” means both dibenzo-α-pyrones of natural origin and synthetic dibenzo-α-pyrones, but also all the derivatives of dibenzo-α-pyrones.

In the sense of the present invention, an active substance, an active principle or an active ingredient means a bioactive substance (a bioactive compound) which is included in the composition of a medicine or a food supplement or any other administrable form to a living being and which gives this medicine, this food supplement or any other form administrable to a living being its therapeutic or preventive properties.

It is recognized that natural or synthetic N-alkylamides are compounds that are very little or even completely insoluble in water, wherein they disperse only weakly or not at all, these compounds therefore having very low bioavailability/bioaccessibility.

However, despite their weak bioavailability/bioaccessibility (i.e. despite the small fraction of the administered dose which actually reaches the blood circulation in unchanged form) but also despite their low solubility and/or despite their low dispersion, particularly in the intestinal medium, the positive effects of natural or synthetic N-alkylamides on various pathologies make them molecules of interest for administration to humans and/or for veterinary use.

Unfortunately, it appears that the current formulations/compositions comprising natural or synthetic N-alkylamides are unsuitable due to their too low or even non-solubility and/or due to their too weak or even non-dispersion in the aqueous phase and/or due to the low release of these compounds from these formulations/compositions, which ultimately results in a low bioavailability/bioaccessibility of these molecules of interest. It must be noted that it also appears that the current manufacturing processes for these formulations are restrictive and difficult to implement.

By the terms “dispersion in an aqueous phase (in an aqueous medium)”, it is understood, within the meaning of the present invention, a system composed of two phases, wherein one of the two phases, called dispersed phase, is finely distributed in the other, called the dispersing phase. This dispersion can be molecular (solution), colloidal (dispersion of submicron particles) or coarser (dispersion of particles greater than 1 μm). More specifically, according to the invention, the term “aqueous phase dispersion” means suspensions, consisting of a solid phase dispersed in an aqueous phase (liquid).

For the purposes of the present invention, the term “solubility” means the ability of a substance, called solute, to dissolve in another substance, called solvent, to form a homogeneous mixture called solution.

The invention aims to overcome the disadvantages of the prior art by providing (1) a composition comprising at least one natural or synthetic dibenzo-α-pyrone as active substance, of which the solubility and/or the dispersion in the aqueous phase (aqueous medium) is increased such that the bioavailability of these compounds is significantly increased and (2) a process for manufacturing such a composition which is easy to implement, which is flexible, which is economically profitable and which ensures that said at least one natural or synthetic dibenzo-α-pyrone as active substance is present and distributed homogeneously in the final composition obtained.

Moreover, the invention intends to provide a composition which is stable over time, i.e. which retains its properties in terms of solubility and/or dispersion of said at least one natural or synthetic dibenzo-α-pyrone as active substance and which retains its properties in terms of rate of release of this compound over time from a composition (formulation) according to the invention, this in particular in the aqueous phase and more specifically in the intestinal medium.

To resolve these problems at least partially, according to an embodiment, a composition in the form of a thermoformed extrudate is also provided according to the invention, possibly packaged in pellet, flake, granule, powder, effervescent tablet or not, in injectable solution or not, in drink, in suspension, in gel, in ointment or in any other suitable form allowing administration to an animal or a human being, comprising at least one natural or synthetic dibenzo-α-pyrone as active substance and at least one natural or syntheric support.

The term “possibly” used above means “optionally” or also “in particular”, which does not at all limit the composition according to the invention to one of the forms in which it could be packaged.

Advantageously, according to the invention, said thermoformed extrudate is in a solid form, in particular in a friable/brittle form.

Preferably, according to the invention, said thermoformed extrudate is friable/brittle. In other words, preferably, according to the invention, said composition is in the form of a friable/brittle thermoformed extrudate.

By the terms “friable/brittle thermoformed extrudate”, it is understood, in the sense of the present invention, an extrudate which disintegrates easily, which can be easily reduced to powder and/or in small fragments/pieces.

The term “support” means, in the sense of the present invention, a compound or a molecule, in particular a compound or a molecule which allows dispersion/solubilization/the integration of the active ingredient (dibenzo-α-pyrone) within it so as to form a mixture/a homogeneous dispersion.

Advantageously, according to the invention, said at least one natural or synthetic support is a thermoplastic support, i.e. a support having the property of softening when it is heated sufficiently, but, as it cools, becomes hard and friable/brittle again.

Advantageously, according to the invention, said natural or synthetic support can be a polymer, in particular a thermoplastic polymer.

Advantageously, according to the invention, the term “support” means the molecules/compounds of polymer type (for example, polysaccharides) or not (for example, amino acids).

Advantageously, according to the invention, said natural or synthetic support is chosen from the group consisting of amino acids, peptides and natural or synthetic polypeptides, natural or synthetic proteins, natural or synthetic saccharides, natural or synthetic lipids, synthetic polymers, urea, their derivatives and their mixtures.

Advantageously, according to the invention, said natural or synthetic support is an amino acid chosen from the group consisting of the following amino acids: aspartic acid, cysteic acid, muramic acid, pyroglutamic acid, alanine, arginine, asparagine, aspartate, citrulline, cysteine, glutamate, glutamine, glycine, histidine, homocysteine, homoserine, hydroxyproline, isovaline, isoleucine, leucine, lysine, methionine, methylalanine, norleucine, norvaline, ornithine, phenylalanine, proline, sarcosine, serine, threonine, tryptophan, tyrosine, valine, their derivatives and their mixtures. This list is not exhaustive.

Advantageously, according to the invention, said natural or synthetic support is a peptide or a polypeptide chosen from the group consisting of aspartame, carnosine, gluthation, collagen peptides, protein hydrolysates, their derivatives and their mixtures. This list is not exhaustive.

In the sense of the present invention, the term “protein” means proteins, hydrolyzed or not, for example, plant or animal proteins, hydrolyzed or not.

Advantageously, according to the invention, said natural or synthetic support is a protein (hydrolyzed or not) chosen from the group consisting of the following proteins: caseins, glycoproteins, collagens, collagen hydrolysates, gelatins, pea proteins, soy protein, wheat protein, pumpkin protein, nut protein, rice protein, hazelnut protein, milk protein, pea protein, lentil protein, almond protein, pistachio proteins, chia proteins, hemp proteins, flax proteins, squash proteins, sesame proteins, poppy proteins, lupine proteins, sunflower proteins, oat proteins, millet proteins, barley proteins, rye proteins, corn proteins, zein, their derivatives and their mixtures.

For example, when said at least one protein is collagen or gelatin, it can be collagen or gelatin of animal origin (fish, pork, beef, etc.).

In particular and advantageously according to the invention, said collagen hydrolysates are collagen hydrolysates not having the capacity to gel, i.e. non-gelling collagen hydrolysates.

The term “protein” means a macromolecule consisting of different amino acids which are bonded together. Protein is the most common element and the basic molecular unit of all living things. Proteins are characterized by the length of their molecular chain.

The term “amino acid” means a carboxylic acid which also has an amine functional group and the term “peptide” means a polymer of amino acids.

The term “collagen” means a protein composed of three associated alpha polypeptide chains. These chains are connected by hydrogen bonds between hydroxylysine and hydroxyproline and covalent bonds.

The term “gelatin” means collagen which has been modified, the molecular chains preferably remaining long enough to ensure the functional properties sought with gelatin (viscosity, hold of the jelly).

The term “collagen peptides” means small ordered groups of amino acids resulting from the fragmentation of the collagen molecule. Depending on the hydrolysis technique used, these groups are more or less important and more or less active.

The term “hydrolyzed collagen” indicates that the collagen has been hydrolyzed, in particular to make it more assimilable.

Preferably, according to the invention, said collagens and/or said collagen hydrolysates and/or said gelatins and/or said protein hydrolystates (or said hydrolyzed proteins) have a molecular weight of between 50 and 300000 Da, preferably between 100 and 275000 Da, preferably between 150 and 250000 Da, preferably between 200 and 225000 Da, preferably between 250 and 200000 Da, preferably between 300 and 175000 Da, preferably between 350 and 150000 Da, preferably between 400 and 125000 Da, preferably between 450 and 100000 Da, preferably between 500 and 75000 Da, preferably between 550 and 50000 Da, preferably between 600 and 40000 Da, preferably between 650 and 30000 Da, preferably between 700 and 20000 Da, preferably between 750 and 10000 Da, preferably between 800 and 9000 Da, preferably between 850 and 8000 Da, preferably between 900 and 7000 Da, preferably between 950 and 6000 Da, preferably between 1000 and 5000 Da, preferably between 1050 and 4000 Da, preferably between 1100 and 3000 Da, preferably between 1150 and 2000 Da, preferably between 1200 and 1000 Da.

Advantageously, according to the invention, said collagens and/or said collagen hydrolysates and/or said gelatins have a molecular weight of between 1000 and 300000 Da, preferably between 1500 and 150000 Da, preferably between 2000 and 60000 Da.

Preferably, according to the invention, said collagens and/or said collagen hydrolysates have a molecular weight equal to 50 Da or equal to 100 Da or equal to 150 Da or equal to 200 Da or equal to 250 Da or equal to 300 Da or equal to 350 Da or equal to 400 Da or equal to 450 Da or equal to 500 Da or equal to 550 Da or equal to 600 Da or equal to 650 Da or equal to 700 Da or equal to 750 Da or equal to 800 Da or equal to 850 Da or equal to 900 or equal to 950 Da or equal to 1000 Da or equal to 1100 Da or equal to 1200 or equal to 1300 Da or equal to 1400 Da or equal to 1500 Da or equal to 1600 Da or equal to 1700 Da or equal to 1800 Da or equal to 1900 Da or equal to 2000 Da or equal to 2500 Da or equal to 3000 Da or equal to 3500 Da or equal to 4000 Da or equal to 4500 Da or equal to 5000 Da or equal to 5500 Da or equal to 6000 Da or equal to 6500 Da or equal to 7000 Da or equal to 7500 Da or equal to 8000 Da or equal to 8500 Da or equal to 9000 Da or equal to 9500 Da or equal to 10000 Da or equal to 12500 Da or equal to 15000 Da or equal to 17500 Da or equal to 20000 Da or equal to 22500 Da or equal to 25000 Da or equal to 27500 Da or equal to 30000 Da or equal to 32500 Da or equal to 35000 Da or equal to 37500 Da or equal to 40000 Da or equal to 42500 Da or equal to 45000 Da or equal to 47500 Da or equal to 50000 Da or equal to 55000 Da or equal to 60000 Da or equal to 65000 Da or equal to 70000 Da or equal to 75000 Da or equal to 80000 Da or equal to 85000 Da or equal to 90000 Da or equal to 100000 Da or equal to 110000 Da or equal to 120000 Da or equal to 130000 Da or equal to 140000 Da or equal to 150000 Da or equal to 160000 Da or equal to 170000 Da or equal to 180000 Da or equal to 190000 Da or equal to 200000 Da or equal to 210000 Da or equal to 220000 Da or equal to 230000 Da or equal to 240000 Da or equal to 250000 Da or equal to 260000 Da or equal to 270000 Da or equal to 280000 Da or equal to 290000 Da or equal to 300000 Da.

According to an embodiment according to the invention, when said at least one protein is collagen, the latter has a molecular weight of between 900 and 7000 Da, preferably a molecular weight of between 950 and 5000 Da, preferably a molecular weight of between 1000 and 3000 Da.

According to an embodiment according to the invention, when said at least one protein is collagen, the latter has a molecular weight equal to 2000 Da or equal to 3000 Da or equal to 5000 Da or equal to 50000 Da.

According to an embodiment according to the invention, when said at least one protein is gelatin, the latter has a molecular weight of between 900 and 6000 Da, preferably a molecular weight of between 950 and 5000 Da, preferably a molecular weight of between 1000 and 3000 Da.

According to an embodiment according to the invention, when said at least one protein is gelatin, the latter has a molecular weight equal to 2000 Da or equal to 3000 Da or equal to 5000 Da or equal to 50000 Da.

According to an embodiment according to the invention, when said at least one protein is a hydrolyzed collagen or a collagen peptide, the latter has a molecular weight of between 900 and 6000 Da, preferably a molecular weight of between 950 and 5000 Da, preferably a molecular weight of between 1000 and 3000 Da.

According to an embodiment according to the invention, when said at least one protein is a hydrolyzed collagen or a collagen peptide, the latter has a molecular weight equal to 2000 Da or equal to 3000 Da or equal to 5000 Da or equal to 50000 Da.

Advantageously, according to the invention, said at least one saccharide is chosen from the group consisting of the following saccharides: raffinose, rhamminose, mannose, glucose, galactose, fructose, rhamnose, saccharose, stachyose, verbascose, trehalose, lactose, lactulose, maltose, erythritol, mannitol, sorbitol, xylitol, cyclodextrines (α-cyclodextrine, β-cyclodextrine, γ-cyclodextrine), isomalt, xylane, alginates, starches (corn starch, potato starch, rice starch, wheat starch, buckwheat starch, chestnut starch, sweet potato starch, pea starch, or also cassava starch), pregelatinized starches, modified starches, soluble or insoluble fibers (Konjac fibers, acacia fibers, inulin fibers, chicory fibers or also arabinoxylane fibers), carrageenans (κ-carrageenan, ι-carrageenan, λ-carrageenan), pectin, hydroxypropylcellulose (HPC), hydroxypropylmethylcellulose (HPMC), ethylcellulose (EC), methylcellulose (MC), hydroxypropymthylcellulose acetosuccinate, cellulose acetobutyrate, cellulose acetophthalate, glycogen, β-glucane, amylopectin, amylose, dextrines, maltodextrine, isomaltose, xylane, pullulan, agar-agar, mannanes, fucoidane, xanthane gum, guar gum, Arabic gum, acacia gum, adragante gum, ghatti gum, mastic gum, karaya gum, Konjac gum, fenugrec gum, mahogany or cashew gum, tara gum, carob gum, cassia gum, benzoin gum, gaiac gum, chitosan, chitin, levan, neoserin, hyaluornic acid, hyaluronates, chondroitin sulfate, dermatane sulfate, keratane sulfate, their derivatives and their mixtures.

As an example, with regard to alginates, calcium alginate, sodium alginate and ammonium alginate may be mentioned. This list is not exhaustive.

In the sense of the present invention, the term “saccharide” groups together monosaccharides, oligosaccharides and polysaccharides.

As an example, among the soluble or insoluble fibers, Konjac fibers, acacia fibers, inulin fibers, chicory fibers or even arabinoxylan fibers can be mentioned. This list is not exhaustive.

As an example, among the dextrins, pea dextrins, potato dextrins, corn dextrins, their derivatives and their mixtures can be mentioned. This list is not exhaustive.

As an example, among the cyclodextrins, α-cyclodextrin, β-cyclodextrin, γ-cyclodextrin, their derivatives and their mixtures can be mentioned. This list is not exhaustive.

As an example, among the hyaluronates, hyaluronic acid, sodium hyaluronate, their derivatives and their mixtures can be mentioned. This list is not exhaustive.

As an example, among the carrageenans, κ-carrageenan, ι-carrageenan, λ-carrageenan, their derivatives and their mixtures can be mentioned. This list is not exhaustive.

As an example, among the starches, corn starch, potato starch, rice starch, wheat starch, buckwheat starch, chestnut starch, sweet potato starch or even cassava starch can be mentioned. This list is not exhaustive.

Advantageously, according to the invention, said natural or synthetic support is a lipid chosen from the group consisting of the following lipids: lecithins, beeswax, candelilla wax, rice bran wax, carnauba wax, animal or plant oils, hydrogenated animal or plant oils, phospholipids, ceramides, fatty acid esters, their derivatives and their mixtures. This list is not exhaustive.

Advantageously, according to an embodiment, said natural or synthetic support is a synthetic polymer chosen from the group consisting of the following synthetic polymers: polyvinyl acetate, polyvinylpyrrolidone (PVP), polyvinylpyrrolidone-co-acetate vinyl, polyethylene-co-vinyl acetate, co-methacrylic polyvinyl acid acetate, polyethylene oxide, polylactide-co-glycolide, polyvinyl alcohol, polycarbophil, polycaprolactone, ethylene-vinyl copolymer, their derivatives and their mixtures. This list is not exhaustive.

The term “extrudate” means, in the sense of the present invention, a material which exits from an apparatus, in particular, an extruder, in particular from the die of an extruder.

The term “thermoformed extrudate” means, in the sense of the present invention, a material which exits from an apparatus, in particular which exits from an extruder, wherein it has undergone a transformation by the effect of the heat, possibly by the combined effect of heat and shear forces of a worm screw. Such a transformation by the effect of heat, possibly by the combined effect of heat and shear forces of a worm screw, can be obtained with the hot melt extrusion (HME) technique.

A thermoformed extrudate according to the invention can therefore be obtained using the hot melt extrusion technique which makes it possible to achieve the molecular dispersion of an agent (active substance) within a support (for example, within a polymer/a polymeric matrix) to form solid dispersions, the agent and/or the support used undergoing a change in state of matter. The agent and/or the support therefore passes from a first state of matter to a second state of matter, which second state of matter implying that said agent and/or said support is at least partially in amorphous form. This solid dispersion and this change of state of the material are possible thanks to an input of heat and possibly thanks to the stress applied by the movement of the worm screws on the material in an extruder. Preferably, hot melt extrusion leads to, at the outlet, the formation of a thermoformed extrudate, in particular to the formation of a friable/brittle thermoformed extrudate, in the form of a rush which can then be pelletized or crushed.

In particular, a thermoformed extrudate according to the invention is an extrudate, wherein at least one natural or synthetic dibenzo-α-pyrone as active substance and/or at least one natural or synthetic support has/have been melted to lead to a solid dispersion, consisting of a glassy structure comprising a molecular mixture of said at least one natural or synthetic dibenzo-α-pyrone as active substance and of said at least one natural or synthetic support such that said at least one natural or synthetic dibenzo-α-pyrone as active substance is dispersed within said at least one natural or synthetic support.

By the term “glassy structure”, it is understood, in the sense of the present invention, a structure comprising/being formed by a molecular mixture of at least two compounds/molecules, at least one of these two compounds/molecules presenting itself at least partially in a non-crystalline form, in particular presenting itself at least partially in amorphous form. In particular, in the scope of the present invention, such a glassy structure is obtained by a change of state of the material comprising the active principle/the active substance (dibenzo-α-pyrone) and/or the support used, which change of state of the material is obtained by hot melt extrusion, the material being subjected to heating and undergoing melting.

By the term “solid dispersion”, it is understood, in the sense of the present invention, a mixture/a system of at least two compounds/molecules, at least one of these two compounds/molecules presenting itself at least partially in a non-crystalline form, in particular presenting itself at least partially in amorphous form.

In particular, a “solid dispersion” consists of a molecular dispersion of an active principle/of an active substance at least partially in amorphous form within a support, for example, within a polymeric matrix.

In particular, a thermoformed extrudate according to the invention, possibly packaged in pellet, flake, granule, powder, effervescent tablet or not, in injectable solution or not, in drink, in suspension, in gel, in ointment or even in any other suitable form allowing administration to an animal or a human being, is an extrudate, wherein the active principle/the active substance (dibenzo-α-pyrone) and/or said at least one support is/are melted.

In particular, the composition according to the invention, more specifically the composition in the form of a thermoformed extrudate according to the invention, possibly packaged in pellet, flake, granule, powder, effervescent tablet or not, in injectable solution or not, in drink, in suspension, in gel, in ointment or in any other suitable form allowing administration to an animal or a human being, is a solid dispersion, wherein said at least one natural or synthetic dibenzo-α-pyrone as active substance is dispersed in said at least one natural or synthetic support.

A thermoformed extrudate according to the invention, possibly packaged in pellet, flake, granule, powder, effervescent tablet or not, in solution for injection or not, in drink, in suspension, in gel, in ointment or in any other suitable form allowing a administration to an animal or a human being, is advantageously obtained by hot thermoforming, in particular by hot thermoforming by the hot melt extrusion technique. Advantageously, according to the invention, hot thermoforming therefore relates more specifically to the hot melt extrusion technique.

Advantageously, according to the invention, hot melt extrusion causes the agent (dibenzo-α-pyrone) and/or the natural or synthetic support to be (preferably simultaneously) mixed, heated, melted, homogenized and extruded. Intense mixing and forced stirring in particular by the worm screws during the hot melt extrusion process causes the disintegration/distribution of the agent particles in the molten natural or synthetic support leading to a solid dispersion. The material (agent+support of natural origin) is ultimately in a “glassy state” or “glassy structure” or vitreous state and behaves like a friable/brittle solid, but without a totally crystalline structure and having a weak arrangement.

Preferably, a composition is therefore provided according to the invention, obtained by hot thermoforming in the form of a thermoformed extrudate, in particular in the form of a friable/brittle thermoformed extrudate, possibly packaged in pellet, flake, granule, powder, effervescent tablet or not, in solution for injection or not, in drink, in suspension, in gel, in ointment or in any other suitable form allowing administration to an animal or a human being, obtained by hot thermoforming, in particular obtained by hot melt extrusion, said composition comprising at least one natural or synthetic dibenzo-α-pyrone as active substance and at least one natural or synthetic support.

A thermoformed extrudate according to the invention, in particular a friable/brittle thermoformed extrudate according to the invention, possibly packaged in pellets, flakes, granules, powders, effervescent tablets or not, in injectable solutions or not, in drink, in suspensions, in gel, in ointment or in any other suitable form allowing administration to an animal or a human being, can therefore be obtained using the hot melt extrusion technique which makes it possible to achieve the molecular dispersion of an agent (active substance) within a support, in particular within of a polymer matrix (within a polymer), to form solid dispersions. This solid dispersion is possible thanks to a heat input and possibly thanks to the stress applied by the movement of the worm screws on the material in an extruder. Ultimately, generally, hot melt extrusion leads to, at the outlet, the formation of a friable/brittle thermoformed extrudate in the form of a rush which can then, in particular, be pelletized or crushed.

The hot melt extrusion technique can be carried out without providing a liquid phase, but relies on providing heat (heating) to ensure a transformation of the material (change of state of the material) by thermoforming. The hot melt extrusion technique leads to a transformation of the material (change of state of the material), in particular to a glassy structure (“glassy structure”) obtained under the action of heat (heating), the particles constituting the powders are no longer all present in their initial (native) crystalline form at the end of the hot melt extrusion process but have undergone transformation by thermoforming.

According to the invention, preferably, said at least one natural or synthetic dibenzo-α-pyrone as active substance comprises at least a first amorphous phase and possibly a second crystalline phase.

By the term “comprises at least a first amorphous phase and possibly a second crystalline phase”, it is understood, in the sense of the present invention, that said at least one natural or synthetic dibenzo-α-pyrone as active substance can either comprise 100% by mass of an amorphous phase, or that it can simultaneously comprise a first amorphous phase and a second crystalline phase, the sum of the percentages by mass of the first and second phases being in this case equal to 100. In other words, the composition according to the invention can comprise said at least one natural or synthetic dibenzo-α-pyrone as active substance (1) totally in amorphous form or (2) partially in amorphous (phase) form and partially in crystalline (phase) form.

It is noted that a phase is called amorphous when the atoms constituting it do not respect any order at medium or long distances, which distinguishes it from a so-called crystalline phase.

In the scope of the present invention, a differential scanning calorimetry (DSC) analysis which is well known to a person skilled in the art can be used to measure the difference in heat exchange between a reference and a product studied. This technique makes it possible to determine the melting points, the crystallization temperatures or the phase transition temperatures, but also the reaction enthalpies. In particular, such an analysis makes it possible to obtain curves where the presence of an endothermic peak (heat absorption) shows the melting of the sample analyzed. A melting peak indicates the presence of a crystalline form in the sample. On the contrary, when the melting peak is not present, the material is in an amorphous state.

The composition according to the invention is therefore preferably in the form of a thermoformed extrudate, possibly packaged in pellet, flake, granule, powder, effervescent tablet or not, in solution for injection or not, in drink, in suspension, in gel, in ointment or in any other suitable form allowing administration to an animal or a human being, wherein said at least one natural or synthetic dibenzo-α-pyrone as active substance comprises at least a first amorphous phase and possibly a second crystalline phase, which phase(s) is/are dispersed within at least one natural or synthetic support.

It has been determined, in the scope of the present invention, that such a composition in the form of a thermoformed extrudate, possibly packaged in pellet, flake, granule, powder, effervescent tablet or not, in injectable solution or not, in drink, in suspension, in gel, in ointment or in any other suitable form allowing administration to an animal or a human being, comprising at least one natural or synthetic dibenzo-α-pyrone as active substance and at least one natural or synthetic support, has a significantly greater solubility and/or dispersion in the aqueous phase (aqueous medium) of said at least one natural or synthetic dibenzo-α-pyrone as active substance. Moreover, it has been shown that such a composition according to the invention has a significantly increased bioavailability/bioaccessibility of said at least one natural or synthetic dibenzo-α-pyrone as active substance compared to bioavailabilities/bioaccessibilities observed for current compositions comprising at least one natural or synthetic dibenzo-α-pyrone.

According to the invention, the active principle/active substance, i.e. said at least one natural or synthetic dibenzo-α-pyrone, is dispersed/spread/distributed homogeneously within at least one natural or synthetic support, the active principle and/or said at least one natural or synthetic support being melted during the thermoforming manufacturing process implemented according to the invention and described below.

Furthermore, a composition according to the invention can be stored for several months without its properties being altered. In particular, it has been demonstrated that a composition according to the invention retains its properties in terms of solubility and/or dispersion in the aqueous phase (aqueous medium) of said at least one natural or synthetic dibenzo-α-pyrone as active substance and in terms of the rate of release of this compound over time from a composition/formulation according to the invention, in particular in the aqueous phase.

Advantageously, according to the invention, said thermoformed extrudate, possibly packaged in pellet, flake, granule, powder, effervescent tablet or not, in injectable solution or not, in drink, in suspension, in gel, in ointment or in any other suitable form allowing administration to an animal or a human being, comprises at least one natural or synthetic dibenzo-α-pyrone as active substance and at least one natural or synthetic support.

Alternatively, according to the invention, said thermoformed extrudate, possibly packaged in pellet, flake, granule, powder, effervescent tablet or not, in injectable solution or not, in drink, in suspension, in gel, in ointment or in any other suitable form allowing administration to an animal or a human being, consists of a thermoformed mixture of said at least one natural or synthetic dibenzo-α-pyrone as active substance and at least one natural or synthetic support.

According to the invention, a thermoformed extrudate is therefore provided, possibly packaged in pellet, flake, granule, powder, effervescent tablet or not, in solution for injection or not, in drink, in suspension, in gel, in ointment or also in any other suitable form allowing administration to an animal or a human being, obtained by hot thermoforming, in particular obtained by hot melt extrusion, said thermoformed extrudate comprising a thermoformed mixture of at least one natural or synthetic dibenzo-α-pyrone as active substance and at least one natural or synthetic support.

By the term “thermoformed mixture”, it is understood, in the sense of the present invention, a mixture which exits from an apparatus, in particular which exits from an extruder, wherein it has undergone a transformation by the effect of the heat, possibly by the combined effect of heat and shear forces of a worm screw. Such a transformation by the effect of heat, possibly by the combined effect of heat and shear forces of a worm screw, can be obtained with the hot melt extrusion (HME) technique.

In particular, a thermoformed mixture according to the invention is a mixture, wherein the active principle/the active substance (dibenzo-α-pyrone) and/or said at least one natural or synthetic support is/are melted/has/have been melted.

A thermoformed mixture according to the invention can therefore be obtained using the hot melt extrusion technique which makes it possible to achieve the molecular dispersion of an agent (active substance) within a natural or synthetic support, for example within a polymeric matrix (within a polymer) to form solid dispersions, the agent and/or the natural or synthetic support implemented undergoing a change in the state of the matter. The agent and/or the natural or synthetic support therefore passes from a first state of matter to a second state of matter, which second state of matter implying that said agent and/or said natural or synthetic support is at least partially in amorphous form.

Preferably, according to the invention, said at least one natural or synthetic dibenzo-α-pyrone as active substance comprises a first amorphous phase.

Advantageously, according to the invention, said at least one natural or synthetic dibenzo-α-pyrone as active substance mainly comprises at least one first amorphous phase.

Preferably, according to the invention, said at least one natural or synthetic dibenzo-α-pyrone as active substance comprises between 51 and 100% by mass of an amorphous phase and between 0 and 49% of a crystalline phase.

By the term “predominantly at least one first amorphous phase”, it is therefore understood, in the sense of the present invention, that said at least one natural or synthetic dibenzo-α-pyrone as active substance comprises between 50 and 100% by mass of a amorphous phase and between 0 and 50% of a crystalline phase, more specifically that said at least one natural or synthetic dibenzo-α-pyrone as active substance comprises between 51 and 100% by mass of an amorphous phase and between 0 and 49% of a crystalline phase.

Preferably, according to the invention, said natural or synthetic dibenzo-α-pyrone as active substance is chosen from the group consisting of the following compounds: urolithin A, isourolithin A, urolithin B, urolithin C, urolithin D, urolithin E, urolithin M-5, urolithin M-6, urolithin M-7, isourolithin B, 8-O-methylurolithin A, 8,9-di-O-methylurolithin C, and 8,9-di-O-methylurolithin D. In this sense, advantageously, said natural or synthetic dibenzo-α-pyrone as active substance is a compound, the basic chemical structure of which is as follows (General formula (II))

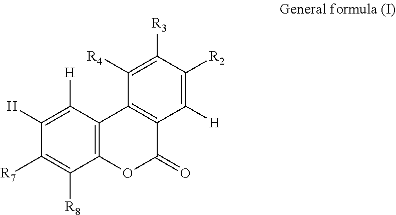

wherein,
R₂ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₃ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₄ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₇ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₈ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucosidev; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;

Advantageously, the composition according to the invention further comprises at least one plasticizing agent. The addition of a plasticizing agent in a composition according to the invention makes it possible to obtain a composition according to the invention through a manufacturing process where temperatures lower than the melting points of said at least one natural or synthetic dibenzo-α-pyrone as active substance and the natural or synthetic support can be used in order to still guarantee a melting of these two compounds and the dispersion/spread/distribution of said at least one natural or synthetic dibenzo-α-pyrone as active substance within the natural or synthetic support.

Preferably, according to the invention, said plasticizing agent is chosen from the group consisting of polyols, lipids, lecithins, sucrose esters, water, triethyl citrate, polyethylene glycol, glycerol, sebate dibutyl, butyl stearate, glycerol monostearate, sodium dodecyl sulfate, diethyl phthalate, their derivatives and their mixtures. This list is not exhaustive.

According to the invention, the preferred plasticizing agents are glycerol, water, polyethylene glycol, triethyl citrate, lecithins, polyols and sucrose esters.

Advantageously, according to the invention, said natural or synthetic dibenzo-α-pyrone as active substance is present in an amount of 1% at 80% by weight relative to the total weight of the composition, preferably at a rate of 10% to 60% by weight relative to the total weight of the composition, preferably at a rate of 15% to 50% by weight relative to the total weight of the composition, preferably at a rate of 20% to 40% by weight relative to the total weight of the composition.

Preferably, according to the invention, said natural or synthetic dibenzo-α-pyrone as active substance is present in an amount of 20% by weight relative to the total weight of the composition, or in an amount of 25% by weight relative to the total weight of the composition, or at a rate of 30% by weight relative to the total weight of the composition, or at a rate of 35% by weight relative to the total weight of the composition, or at a rate of 40% by weight relative to the total weight of the composition.

Preferably, according to the invention, said at least one natural or synthetic support is present in an amount of 5% to 90% by weight relative to the total weight of the composition, preferably at a rate of 15% to 85% by weight relative to the total weight of the composition, preferably at a rate of 20% to 80% by weight relative to the total weight of the composition, preferably at a rate of 50% to 75% by weight relative to the total weight of the composition.

Advantageously, according to the invention, said at least one natural or synthetic support is present at a rate of 5% by weight relative to the total weight of the composition, or at a rate of 10% by weight relative to the total weight of the composition, or at a rate of 15% by weight relative to the total weight of the composition, or at a rate of 20% by weight relative to the total weight of the composition, or at a rate of 25% by weight relative to the total weight of the composition, or at a rate of 30% by weight relative to the total weight of the composition, or at a rate of 35% by weight relative to the total weight of the composition, or at a rate of 40% by weight relative to the total weight of the composition, or at a rate of 45% by weight relative to the total weight of the composition, or at a rate 50% by weight relative to the total weight of the composition, or at a rate of 55% by weight relative to the total weight of the composition, or at a rate of 60% by weight relative to the total weight of the composition, or at a rate of 65% by weight relative to the total weight of the composition, or at a rate of 70% by weight relative to the total weight of the composition, or at a rate of 75% by weight relative to the total weight of the composition.

When it is a mixture of two supports according to the invention, the ratio between these two supports is preferably around 10:90 or around 20:80 or around 30:70 or around 40:60 or around 50:50.

Advantageously, according to the invention, said at least one plasticizing agent is present at a rate of 1% to 30% by weight relative to the total weight of the composition, preferably at a rate of 10% to 25% by weight relative to the total weight of the composition, preferably at a rate of 15% to 20% by weight relative to the weight total composition.

Advantageously, according to the invention, said at least one plasticizing agent is present at a rate of 5% by weight relative to the total weight of the composition, or at a rate of 10% by weight relative to the total weight of the composition, at a rate of 15% by weight relative to the total weight of the composition, at a rate of 20% by weight relative to the total weight of the composition, at a rate of 25% by weight relative to the total weight of the composition, at a rate of 30% by weight relative to the total weight of the composition.

Preferably, the composition according to the invention further comprises at least one additive chosen from the group consisting of lubricating agents, surfactant agents, antioxidant agents, chelating agents, their derivatives and their mixtures.

As an example, the following compounds can be used, individually or in mixture, as lubricating agents in a composition according to the invention: glycerol dibehenate, talc, silica, stearic acid, boric acid, waxes, sodium oleate, sodium acetate, magnesium stearate, calcium stearate, sodium stearate, sodium benzoate, sodium lauryl sulfate, glycerol distearate, palmitostearate glycerol, microcrystalline cellulose or even polyoxyl-8-glycerides.

As an example, the following compounds can be used, individually or in a mixture, as surfactant agents in a composition according to the invention: Pluronic®, Span®, Cremophor®, polysorbates (Tween®, etc.), vitamin E TPGS and sodium ducosate.

As an example, the following compounds can be used, individually or in mixture, as antioxidant agents and/or chelating agents in a composition according to the invention: butylated hydroxytoluene, butylated hydroxyanisiole, EDTA, citric acid, ascorbic acid and vitamin E.

Advantageously, the composition according to the invention further comprises at least one additional polyphenol type compound chosen from the group consisting of phenolic acids, stilbenes, phenolic alcohols, lignans, flavonoids, their derivatives and their mixtures. In particular, the glycosylated and aglycone forms of the polyphenols are envisaged as additional active principle according to the present invention. More specifically, in the sense of the present invention, the term “polyphenol” means both polyphenols of natural origin and synthetic polyphenols, but also all derivatives of polyphenols.

As an example, in the sense of the present invention, the derivatives of hydroxybenzoic acid (gallic acid, tannic acid, etc.) and the derivatives of hydroxycinamic acid (curcumin, coumaric acid, caffeic acid, ferulic acid, etc.) can be cited as phenolic acids.

As an example, in the sense of the present invention, resveratrol, sirtinol, piceatannol or even polydatin can be cited as stilbenes.

As an example, in the sense of the present invention, flavanoles (quercetin, myricetin, kaempferol, isorhamnetin, morin, rutin, tiliroside, trihydroxyethylrutin, fisetin, etc.), flavones (apigenin, luteolin, baicalein, chrysin, diosmin, nobiletin, tangeretin, wogonin, aminogenistein, etc.), flavanones (bavachin, 8-isopentenylnaringenin, isoxanthohumole, naringenin, eriodictyole, hesperetin, silybin, taxifolin, etc.), isoflavones (genistein, daidzein, daidzin, formonetin, genistin, neobviensoflavone, pueranin, etc.), antocianidins (cianidin, pelargonidin, delphinidin, petunidin, malvidin, etc.) and flavanols (cathechins, gallocatechin, epigallocatechingallate, etc.) can be cited as flavanoids.

According to the invention, said at least one additional active principle of polyphenol type constitutes an efflux pump inhibitor/modulator, including the P-gp.

Preferably, the composition according to the invention further comprises at least one inhibitor and/or one modulator of P-gp activity.

Preferably, the composition according to the invention is packaged in the form of pellets, flakes, granules, powders, effervescent tablets or not, injectable solutions or not, suspensions, gels, ointments or even in any other suitable form allowing administration to an animal or a human being.

Other embodiments of a composition according to the invention are indicated in the accompanying claims.

The invention also relates to a manufacturing process, in particular a manufacturing process by thermoforming, of a composition in the form of a thermoformed extrudate according to the invention, characterized in that it comprises the following steps:
a step of simultaneously or deferringly supplying, over time, at least one natural or synthetic dibenzo-α-pyrone as active substance and at least one natural or synthetic support, to provide an extruder,
a step of mixing, in said extruder, said at least one natural or synthetic dibenzo-α-pyrone as active substance and said at least one natural or synthetic support, to form a mixture, and
a step of hot melt extrusion of said mixture obtained in step b) in said extruder to obtain a thermoformed extrudate, in particular to obtain a thermoformed extrudate consisting of a solid dispersion, more specifically to obtain a thermoformed extrudate consisting of a solid dispersion consisting of a glassy structure comprising a molecular mixture of said at least one natural or synthetic dibenzo-α-pyrone as active substance and of said at least one natural or synthetic support.

Preferably, according to the invention, steps a) and b) occur simultaneously.

Preferably, according to the invention, steps a), b) and c) occur simultaneously.

Such a process according to the invention leads to a composition in the form of a thermoformed extrudate, in particular to a composition in the form of a friable/brittle thermoformed extrudate, i.e. obtained by thermoforming and more specifically by hot melt extrusion, wherein said at least one natural or synthetic dibenzo-α-pyrone as active substance preferably comprises at least one first amorphous phase and possibly one second crystalline phase dispersed within said at least one natural or synthetic support.

This composition according to the invention has a clearly greater solubility and/or dispersion in the aqueous phase (aqueous medium) of said at least one natural or synthetic dibenzo-α-pyrone as active substance and simultaneously a significantly increased bioavailability/bioaccessibility of said at least one natural or synthetic dibenzo-α-pyrone as active substance compared to the solubilities and/or to the dispersions and bioavailabilities/bioaccessibilities of these compounds for current compositions. It has been shown that the composition according to the invention is in the form of a thermoformed extrudate, in particular in the form of a friable/brittle thermoformed extrudate, wherein said at least one natural or synthetic dibenzo-α-pyrone as active substance (active principle) preferably comprising at least a first amorphous phase and possibly a second crystalline phase is dispersed within said at least one natural or synthetic support.

It has also been shown, in the scope of the present invention, that said at least one natural or synthetic dibenzo-α-pyrone as active substance is not degraded, or at the very least, that it is only degraded in a lesser measure, even during the manufacturing process by thermoforming of the composition in the form of a thermoformed extrudate, in particular in the form of a friable/brittle thermoformed extrudate, which however implies the subjecting of said at least one natural or synthetic dibenzo-α-pyrone as active substance to high temperatures (HME). Moreover, it has also been highlighted that said at least one natural or synthetic dibenzo-α-pyrone as active substance in a composition in the form of a thermoformed extrudate according to the invention, in particular in the form of a friable/brittle thermoformed extrudate according to the invention, is spread (distributed) homogeneously.

More specifically, hot melt extrusion (HME) carried out according to the thermoforming manufacturing process according to the invention leads to a melting of the active principle/of the active substance (said at least one natural or synthetic dibenzo-α-pyrone) and/or said at least one natural or synthetic support at a temperature greater than or equal to their melting point.

However, according to certain embodiments of a composition according to the invention, this melting of the active principle/of the active substance and the natural or synthetic support can occur at a temperature below their melting point. This is, for example, the case if the composition according to the invention comprises a plasticizing agent or if the active principle/the active substance itself has plasticizing properties. Such melting of the active principle/of the active substance and/or of the natural or synthetic support gives rise to a solid dispersion, wherein the active principle/the active substance (said at least one natural or synthetic dibenzo-α-pyrone) preferably comprising at least a first amorphous phase and possibly a second crystalline phase is dispersed/spread/distributed homogeneously within said at least one natural or synthetic support.

Advantageously, according to the process according to the invention, step c) of hot melt extrusion is a step according to which said natural or synthetic dibenzo-α-pyrone and/or said at least one natural or synthetic support undergo(es) a change of state of matter, said at least one natural or synthetic dibenzo-α-pyrone as active substance and/or said at least one natural or synthetic support passing from a first state of matter to a second state of matter, which second state of matter implying in particular that said at least one natural or synthetic dibenzo-α-pyrone as active substance and/or said at least one natural or synthetic support is/are presented at least partially in amorphous form.

Advantageously, the process according to the invention comprises a prior step of pre-mixing said at least one natural or synthetic dibenzo-α-pyrone as active substance and said at least one natural or synthetic support, so as to form a pre-mixture intended to supply the extruder.

Preferably, according to the process according to the invention, said hot melt extrusion step is carried out at an extrusion temperature or thermoforming temperature of between 20 and 300° C.

Advantageously, according to the process according to the invention, said hot melt extrusion step is carried out at a rotation speed of an extrusion screw of between 20 and 900 rpm, preferably between 50 and 300 rpm, preferably between 100 and 250 rpm, preferably equal to 250 rpm, preferably equal to 200 rpm, preferably equal to 100 rpm.

Preferably, the process according to the invention comprises an additional cooling step at the outlet of the extruder.

Advantageously, the process according to the invention comprises an additional step of processing the thermoformed extrudate, in particular the friable/brittle thermoformed extrudate, at the outlet of the extruder, for example a cutting at a pelletizer and/or a grinding and/or a crushing and/or a reduction into powder (in powder form) of said thermoformed extrudate, in particular of said friable/brittle thermoformed extrudate.

Other embodiments of the process according to the invention are indicated in the accompanying claims.

The present invention also relates to a composition in the form of a thermoformed extrudate, possibly packaged in pellet, flake, granule, powder, effervescent tablet or not, in solution for injection or not, in drink, in suspension, in gel, in ointment or even in any other suitable form allowing administration to an animal or a human being, obtained according to the process according to the invention.

The present invention also relates to a composition in the form of a thermoformed extrudate, in particular in the form of a friable/brittle thermoformed extrudate, possibly packaged in pellet, flake, granule, powder, effervescent tablet or not, in solution for injection or not, in drink, in suspension, in gel, in ointment or in any other suitable form allowing administration to an animal or a human being, obtained according to the process according to the invention, said composition comprising at least one natural or synthetic dibenzo-α-pyrone as active principle/active substance and at least one natural or synthetic support, said at least one natural or synthetic dibenzo-α-pyrone as active substance preferably comprising at least one first amorphous phase and possibly one second crystalline phase.

In other words, the present invention also relates to a composition in the form of a thermoformed extrudate, in particular in the form of a friable/brittle thermoformed extrudate, possibly packaged in pellet, flake, granule, powder, effervescent tablet or not, in solution for injection or not, in drink, in suspension, in gel, in ointment or in any other suitable form allowing administration to an animal or a human being, obtained by hot melt extrusion (HME), said composition comprising at least one natural or synthetic dibenzo-α-pyrone as active principle/active substance and at least one natural or synthetic support, said at least one natural or synthetic dibenzo-α-pyrone as active substance preferably comprising at least one first amorphous phase and possibly one second crystalline phase.

The present invention also relates to a use of a composition according to the invention as a food supplement and/or as a cosmetic product and/or as a medicine for human or veterinary use and/or as a compound administered via a medical device, for example via a spray or via a patch.

In particular, the present invention relates to a composition for use in preventive and/or curative treatment, in humans and/or in animals, of pathologies linked to the muscular and neuromuscular system, pathologies linked to the vertebral column and joints, pathologies linked to the cardiovascular system (ischemia, metabolic disorders, etc.), of pathologies linked to the hormonal system, of pathologies linked to the gastrointestinal system (nausea, vomiting, diarrhea, digestive inflammations, etc.), of pathologies linked to premature aging of cells, antioxidizing, of pathologies linked to the endocrine system (hyperglycemia, etc.), of pathologies linked to the immune system (inflammatory diseases, myopathies, rheumatoid arthritis, multiple sclerosis, osteoarthritis, fibromyalgia, etc.), of pathologies linked to the central nervous system (pain, migraine, epilepsy, Parkinson's disease, Alzheimer's disease, anxiety, depression, etc.), of sleep disorders, of skin diseases, of diseases due to the presence of microorganisms, of cancers (anti-tumor, etc.), of eye diseases (glaucoma, etc.), of diseases of the respiratory system (asthma, etc.) and in preventive and/or curative treatment for diabetes.

More specifically, the present invention relates to a composition for use in preventive and/or curative treatment, in humans and/or in animals, diseases linked to neuropsychiatric disorders, pain, neurodegenerative diseases, inflammatory diseases, metabolic and renal disorders, musculoskeletal disorders, hormonal disorders, cardiovascular diseases, osteoporosis, endometriosis, dermatitis and skin diseases or even irritable bowel syndrome (IBS).

More specifically, the present invention relates to a composition used in humans and/or in animals, as an antioxidant, anti-inflammatory, anticonvulsant, antiemetic, anxiolytic, hypnotic or even as an antipsychotic.

A composition according to the invention preferably has anabolizing, analgesic, relaxing, anesthetic, antithrombotic, antiviral, antifungal and antibacterial, antimalarial, insecticidal, immunomodulatory, hormone-modulating, oestrogen-modulating, androgenic, spermatogenic, antimutagenic, antiepileptic, anti-inflammatory, neuroprotective, anticonvulsant, anxyolitic, antidepressant, antipsychotic, antithrombotic, antitumor, antidiabetic and/or immunoregulatory properties.

Other forms of use of a composition according to the invention are indicated in the accompanying claims.

### EXAMPLES

### Example 1: Manufacturing Process by Thermoforming of Compositions According to the Invention in the Form of Thermoformed Extrudates

Thermoformed compositions according to the invention comprising at least one dibenzo-α-pyrone, such as those forming the subject of Example 2 below, were obtained according to the following process which is also the subject matter of the present invention:
a) a step of pre-mixing at least one dibenzo-α-pyrone in the native state in powder-form and at least one natural or synthetic support;
b) a step of supplying said pre-mixture formed in step a) to supply a Process 11 Hygienic-type extruder from Thermo-Fischer®;
c) a step of mixing, in said extruder, said pre-mixture to obtain a mixture;
d) a thermoforming step by hot melt extrusion of said mixture obtained in step c) in said extruder to obtain a thermoformed extrudate, in particular a friable/brittle thermoformed extrudate, the hot melt extrusion step being carried out at a rotation speed of an extrusion screw of 100 rpm and at a temperature of between 20 and 160° C.;
e) a step of cooling said thermoformed extrudate, in particular said friable/brittle thermoformed extrudate, at the outlet of the extruder, obtained in step d); and
f) a cutting/crushing step, at a crusher, the cooled thermoformed extrudate obtained in step e), in particular the friable/brittle thermoformed extrudate obtained in step e), so as to obtain a homogeneous powder, i.e. a thermoformed extrudate packaged in powder (in powder form).

The thermoforming temperature at which the hot melt extrusion step is carried out is determined by the type of constituents used, in particular depending on the type of support, and/or of plasticizing agent implemented, which a person skilled in the art is able to determine. Moreover, a person skilled in the art, in particular depending on the type of extruder used and according to the general principle of hot melt extrusion (HME), is able to define possible temperature levels in different zones along the extrusion screw(s) such that there is a progressive increase in the temperature within the material transported by the extrusion screw(s), this in a forward direction of the material within the extruder. Typically, between defined zones along the extrusion screw(s), temperature differences of around 0 to 40° C. are observed. For example, in the scope of the present invention, the compositions tested below were obtained in a Process 11 Hygienic-type extruder from Thermo-Fischer® having 8 temperature zones which are as follows in a forward direction of the matter moving at a speed of 100 rpm: 30° C.-60° C.-100° C.-120° C.-140° C.-150° C.-160° C. and 160° C.

### Example 2: Dispersion and Solubility Tests of Thermoformed Compositions According to the Invention

Thermoformed compositions, obtained according to the manufacturing process described in Example 1, have been tested in terms of dispersion and solubility of urolithin A or urolithin B. This dispersion and this solubility have been measured over time at the start of thermoformed extrudates obtained according to the invention. As indicated above, the thermoformed extrudates are in the form of a homogeneous powder (grind), wherein the urlithin A or the urolithin B preferably comprises at least one amorphous phase.

The dispersion and solubility test was carried out with a paddle dissolution apparatus starting from approximately 800 mg of thermoformed extrudate (approximately 160 mg of urolithin A or urolithin B), at a temperature of 37° C. with stirring at 50 rpm in 900 ml of a 0.1N HCl dissolution medium. This dispersion and solubility test was carried out according to the recommendations of the pharmacopoeia Ph.Eur.9.0 (Recommendations on Dissolution Testing). At specified times (after 15 minutes, after 30 minutes, after 1 hour), a 1 ml sample is taken to carry out the dispersion and solubility test.

In order to carry out the dispersion and solubility test, the sample is diluted in an appropriate solvent (mobile phase: methanol mixture (70%)-isopropanol (30%) then filtered through a filter (PET, pore size of 0.45 μm, Macherey Nagel) before HPLC analysis (Nucleodur 100-5 EC C18 column 125/4 (Macherey-Nagel); mobile phase: 70% methanol and 30% isopropanol; flow rate: 0.6 ml/min; loop: 10 μl, t°=25° C.; 210 nm).

The thermoformed compositions according to the invention shown in Tables 1 and 2 were formulated according to the process of the invention and tested in terms of dispersion and solubility over time according to the principle indicated above. Urolithin A or urolithin B in native crystalline form and in powder form (native urolithin A or native urolithin B) has been used as control. The quantities mentioned in Tables 1 and 2 are percentages by weight of the compounds used (subjected to the process according to the invention) relative to the total weight of the composition.

TABLE 1

| | Urolithin A | Protein | Glycerol |
|---|---|---|---|
| | (1) | (2) | (3) |
| Compo 1 | 20 | 70 | 10 |

(1) Urolithin A (Pincredit Bio-Tech)
(2) Collagen peptides (Peptan B) (Rousselot)
(3) Glycerol (Brenntag)

TABLE 2

| | Urolithin B | Protein | Glycerol |
|---|---|---|---|
| | (1) | (2) | (3) |
| Compo 1 | 20 | 70 | 10 |

(1) Urolithin B (Cimasci)
(2) Collagen peptides (Peptan B) (Rousselot)
(3) Glycerol (Brenntag)

The results obtained from the dispersion and solubility tests are presented in figures 1 (urolithin A) and 2 (urolithin B). As can be observed, the dispersion+the solubility measured for urolithin A or urolithin B at the start of compositions in the form of thermoformed extrudates according to the invention (Compo 1) is clearly greater relative to the control (native urolithin A or native urolithin B).

The present invention has been described in relation to specific embodiments, which have purely illustrative value and must not be considered as limiting. Generally, it will appear clear for a person skilled in the art that the present invention is not limited to the examples illustrated and/or described above.

The use of the verbs “comprise”, “include”, “have”, or any other variant, as well as their conjugations, cannot in any way exclude the presence of elements other than those mentioned.

The use of the indefinite article “a”, “an”, or the definite article “the”, to introduce an element does not exclude the presence of a plurality of these elements.

1. A composition in the form of a thermoformed extrudate in any suitable form allowing administration to an animal or a human being, comprising at least one natural or synthetic dibenzo-α-pyrone as active substance and at least one natural or synthetic support.

2. The composition according to claim 1, wherein said at least one natural or synthetic dibenzo-α-pyrone is a compound, of which the basic chemical structure is as follows (General formula (I)): 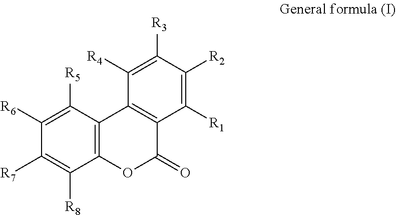
wherein,
R₁ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₂ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₃ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₄ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₅ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₆ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₇ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₈ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl.

3. The composition according to claim 1, wherein said natural or synthetic support is chosen from the group consisting of amino acids, peptides and natural or synthetic polypeptides, natural or synthetic proteins, natural or synthetic saccharides, natural or synthetic lipids, synthetic polymers, urea, their derivatives and their mixtures.

4. The composition according to claim 1, wherein said natural or synthetic dibenzo-α-pyrone is a compound selected from the group consisting of: urolithin A, isourolithin A, urolithin B, urolithin C, urolithin D, urolithin E, urolithin M-5, urolithin M-6, urolithin M-7, isourolithin B, urolithin A glucuronide, urolithin B glucuronide, 8-Omethylurolithin A, 8,9-di-O-methylurolithin C, and 8,9-di-O-methylurolithin D, and combinations thereof.

5. The composition according to claim 4, wherein said at least one of said natural or synthetic dibenzo-α-pyrone is a compound, the basic chemical structure of which is as follows (General formula (II)): 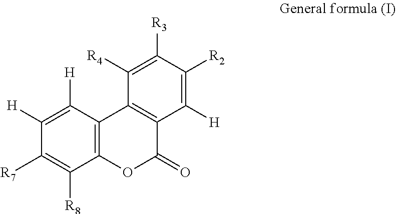
wherein,
R₂ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₃ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₄ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₇ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl;
R₈ represents a hydrogen, a C₁₋₄ alkyl, CH(═O), C(═O)OR′, OR′, OSO₃R′, C(═O)R′, OC(═O)R′, a halogen, an O-dihydrobenzofurane, an O-glucoside, a C-glucoside, an acyl O-glucoside, an acyl C-glucoside, a sulfate O-glucoside, or a sulfate C-glucoside; said C₁₋₄ alkyl being optionally substituted by one or more hydroxys; said O-dihydrobenzofurane being optionally substituted by one or more groups chosen from among OR′ or CH₂OR′; R′ representing a hydrogen or a C₁₋₄ alkyl.

6. The composition according to claim 1, wherein it further comprises at least one plasticizing agent.

7. The composition according to claim 1, wherein it further comprises at least one additive chosen from the group consisting of lubricating agents, surfactant agents, antioxidant agents, chelating agents, their derivatives, and their mixtures.

8. The composition according to claim 1, wherein it further comprises at least one first additional compound of polyphenol type chosen from the group consisting of phenolic acids, stilbenes, phenolic alcohols, lignans, flavonoids, their derivatives and their mixtures.

9. A manufacturing process by thermoforming, of a composition in the form of a thermoformed extrudate according to claim 1, wherein it comprises the following steps:
a) a step of simultaneously or deferringly supplying, over time, at least one natural or synthetic dibenzo-α-pyrone as active substance and at least one natural or synthetic support, to provide an extruder,
b) a step of mixing, in said extruder, said at least one natural or synthetic dibenzo-α-pyrone as active substance and said at least one natural or synthetic support, to form a mixture, and
c) a step of hot melt extrusion of said mixture obtained in step b) in said extruder to obtain a thermoformed extrudate, in particular to obtain a thermoformed extrudate consisting of a solid dispersion, more specifically to obtain a thermoformed extrudate consisting of a solid dispersion consisting of a glassy structure comprising a molecular mixture of said at least one natural or synthetic dibenzo-α-pyrone as active substance and of said at least one natural or synthetic support.

10. The process according to claim 9, wherein it comprises a prior step of pre-mixing said at least one natural or synthetic dibenzo-α-pyrone as active substance and said at least one natural or synthetic support, so as to form a pre-mixture intended to supply the extruder.

11. The process according to claim 9, wherein said hot melt extrusion step is carried out at an extrusion temperature of between 20° C. and 300° C.

12. The process according to claim 9, wherein said hot melt extrusion step is carried out at a rotation speed of an extrusion screw of between 20 and 900 rpm.

13. The process according to claim 9, wherein in that it comprises an additional cooling step at the outlet of the extruder.

14. The process according to claim 9, wherein it comprises an additional step of processing the thermoformed extrudate at the outlet of the extruder.

15. A composition in the form of a thermoformed extrudate in any other suitable form allowing administration to an animal or a human being, according to claim 1 for use in preventive and/or curative treatment, in humans and/or in animals, of pathologies linked to the muscular and neuromuscular system, of pathologies linked to the vertebral column and joints, of pathologies linked to the cardiovascular system, of pathologies linked to the hormonal system, of pathologies linked to the gastrointestinal system, of pathologies linked to premature aging of cells, antioxidizing, of pathologies linked to the endocrine system, of pathologies linked to the immune system, of pathologies linked to the central nervous system, of sleep disorders, of skin diseases, of diseases due to the presence of microorganisms, of cancers, of eye diseases, of diseases of the respiratory system, and in preventive and/or curative treatment for diabetes.

16. A composition in the form of a thermoformed extrudate in any other suitable form allowing administration to an animal or a human being, obtained according to the process according to claim 9.
